(19) United States
(12) Patent Application Publication
(10) Pub. No.: US 2024/0203545 A1
(43) Pub. Date: Jun. 20, 2024

(54) ARTIFICIAL INTELLIGENCE MEDICAL EXAMINATION AND MEDICAL RECORD GENERATION SYSTEM AND METHOD THEREOF

(71) Applicant: BePlus Healthcare Inc., Gangwon-do (KR)

(72) Inventors: Hoon Jae CHUNG, Seoul (KR); Ki Joon HUGH, Busan (KR); Jae Young KIM, Seoul (KR); Jee Hong KIM, Seoul (KR); Woo Taek LIM, Seoul (KR)

(21) Appl. No.: 18/203,795

(22) Filed: May 31, 2023

(30) Foreign Application Priority Data
Dec. 19, 2022 (KR) 10-2022-0178506

(51) Int. Cl. G16H 10/60

(52) U.S. Cl. CPC G16H 10/60

(57) ABSTRACT

An artificial intelligence medical examination and medical record generation system includes a medical examination database storing a medical examination item and a past medical examination result, a medical examination generation unit generating or updating a medical examination scenario by selecting a medical examination item through a medical examination-generation artificial intelligence based on patient data, a medical examination provision unit providing the medical examination scenario with an expression familiar to a patient through natural language processing of a medical examination-provision artificial intelligence based on the patient data, a medical examination analysis unit analyzing a patient answer to the medical examination scenario through structuralizing, standardizing, and encoding processes, a record generation unit generating a medical record familiar to medical personnel based on an analysis result generated by the medical examination analysis unit, and a transmission unit transmitting the medical record to the medical personnel and an electronic medical record system.

### CROSS REFERENCE TO RELATED APPLICATIONS AND CLAIM OF PRIORITY

This application claims benefit of priority to Korean Patent Application No. 10-2022-0178506, filed on Dec. 19, 2022, in the Korean Intellectual Property Office, the disclosure of which is incorporated herein by reference in its entirety.

### BACKGROUND

### 1. Field

The present disclosure relates to a medical examination and medical record generation system, and more particularly, to an artificial intelligence medical examination and medical record generation system which may reconfigure medical examination including a medical term into everyday language and provide the same to a medical consumer, load a database (DB) with structuralized data acquired by structuralizing and standardizing an answer written in everyday language into the medical term, and reconfigure the structuralized data into a medical record based on the medical terms and provided the same to medical personnel, and a method thereof.

### 2. Description of the Related Art

Medical examination indicates a process of acquiring medical data of a patient, such as the main symptom, symptom characteristic, occurrence time, associated symptom, past history and family history of the patient, in the form of questions and answers. Medical personnel may determine urgency of the symptom complained of by the patient through the medical examination, identify a disease requiring a differential diagnosis, and establish a plan for future diagnosis and therapy. However, the data acquired through the medical examination in a clinical field so far is unstructured medical data freely recorded in an arbitrary format by the medical personnel such as doctors and nurses. Accordingly, there are many difficulties in processing such data into meaningful data and using the same for a statistical analysis or the like.

Meanwhile, importance of various medical data necessary for the development of an artificial intelligence has emerged as attempts to introduce the artificial intelligence to a medical field are increased to provide the patient with more optimized medical care. However, the medical examination data are not properly utilized although these data are important data playing a key role in early clinical determination because it takes a lot of time and cost to secure dedicated personnel for standardizing and refining the data for the data to be used for the development of the artificial intelligence.

In addition, an amount of time the medical personnel may devote to an individual patient is gradually decreasing as medical use is rapidly increased due to a social change such as population aging. According to a recent survey, 61% of doctors answered that ‘treatment time is insufficient’ as a result of the survey conducted on a total of 1,200 people including general practitioners and specialists. In a situation where as much patient data as possible needs to be collected within a limited time, patient satisfaction with a medical service may be lower, and a misdiagnosis may also occur due to the short treatment time.

There is an attempt to provide the patient with the medical examination in advance through a medium such as a paper or web page in order to utilize the limited treatment time more efficiently by collecting the data that the medical personnel basically needs for the treatment in advance. In addition, it is also meaningful in that the unstructured medical examination data may be collected in a standardized form through this attempt. However, such a conventional medical examination system may often use a method of requiring a short answer such as yes/no, thus making it difficult to collect specific data from the patient, and may usually include an expression that is easy for the medical personnel to understand, thus making patient understanding lower.

### SUMMARY

An aspect of the present disclosure is to provide a remote interactive medical examination and medical record generation system to promote a medical consumer to correctly identify his/her condition and appropriately use a medical service by providing his/her specific data, and to help medical personnel efficiently use limited medical resources by quickly identifying a patient condition.

Another aspect of the present disclosure is to help the patient have a better understanding and comfortably perform medical examination by using an artificial intelligence to select the medical examination appropriate for a patient symptom, and reconfigure the medical examination into an easy-to-understand expression used in everyday life just like the patient consultations with a real doctor.

Another aspect of the present disclosure is to help medical personnel naturally understand a medical examination result and provide a patient with effective treatment counseling by using an artificial intelligence to reconfigure a patient answer to medical examination into a medical record in the form of a treatment chart written based on a medical term familiar to medical personnel.

Another aspect of the present disclosure is to help medical personnel easily check a medical examination result through a system the medical personnel usually use, and to save time necessary for organizing and inputting the medical examination result by transmitting a generated medical record to a medical personnel terminal or an electronic medical record (EMR) system accessed by the medical personnel.

Another aspect of the present disclosure is to provide medical data acquired through a medical examination system with higher efficiency and interoperability by structuralizing and standardizing the components and results of medical examination by the medical examination system using ‘systematized nomenclature of medicine clinical terms (SNOMED-CT)’ which are international standard medical terms and ‘Korean standard terminology of medicine (KOSTOM)’ which is a Korean standard medical terminology developed by the Ministry of Health and Welfare in Korea.

In an aspect, an artificial intelligence medical examination and medical record generation system may include: a medical examination database (DB) storing a medical examination item and a past medical examination result: a medical examination generation unit generating or updating a medical examination scenario by selecting an appropriate medical examination item through a medical examination-generation artificial intelligence based on patient data: a medical examination provision unit processing and providing the medical examination scenario with an expression familiar to a patient through natural language processing of a medical examination-provision artificial intelligence based on the patient data: a medical examination analysis unit analyzing a patient answer to the medical examination scenario through structuralizing, standardizing, and encoding processes: a record generation unit generating a medical record familiar to medical personnel based on an analysis result generated by the medical examination analysis unit; and a transmission unit transmitting the medical record to the medical personnel and an electronic medical record (EMR) system.

The medical examination analysis unit may include: a structurer classifying the answer to the medical examination item into an item or attribute necessary for a diagnosis based on its meaning: a standardizer replacing the classified item and attribute with a standard word or sentence having the same meaning in standard terminology using the Korean standard terminology of medicine (KOSTOM) or systematized nomenclature of medicine clinical terms (SNOMED-CT); and an encoder assigning a code corresponding to the standard terminology to the standard word or sentence.

The medical examination generation unit may generate the medical examination scenario by selecting the appropriate medical examination item among the stored medical examination items based on the patient data and a patient symptom.

The patient data may include data on a past treatment history of the patient, and one or more of age, gender, usage language, education level, past treatment history, residential area, lifestyle, or allergy data, input by the patient, before medical examination is generated, and the medical examination generation unit may update the medical examination item in consideration of a corrected content when the patient data or data acquired by structuralizing an existing medical examination item and an answer to the medical examination item is corrected.

The medical examination generation unit may generate the updated medical examination scenario by selecting an additionally-necessary medical examination item based on the analysis result generated by the medical examination analysis unit when the patient inputs the answer to the medical examination scenario.

The medical examination provision unit may convert an expression in the medical examination scenario into a familiar expression that is easy for the patient to understand through the natural language processing based on the patient data.

The record generation unit may generate the medical record by converting the patient answer to the medical examination scenario into a form that is easy for the medical personnel to understand based on the analysis performed by the medical examination analysis unit.

The transmission unit may directly transmit the medical record to a medical personnel terminal or to an EMR server of the linked EMR system to which the medical personnel have access rights.

In another aspect, an artificial intelligence medical examination and medical record generation method may include: a) analyzing data of a patient who wants to use an artificial intelligence medical examination and medical record generation system: b) generating a medical examination scenario by selecting a medical examination item corresponding to the patient data: c) reconfiguring the medical examination scenario into an expression corresponding to the patient data and providing the expression to the patient: d) systematizing and analyzing an answer to the medical examination scenario that is received from the patient: e) generating a medical record for the patient based on an analysis result of the answer to the medical examination scenario; and f) transmitting the medical record to a medical personnel terminal or a linked electronic medical record (EMR) server.

The patient data may include data on a past treatment history of the patient, and one or more of age, gender, usage language, education level, past treatment history, residential area, lifestyle, or allergy data, and in the step b), the medical examination item may be corrected or updated in consideration of a corrected content when the patient data or data acquired by structuralizing an existing medical examination item and an answer to the existing medical examination item is corrected.

In the step b), the updated medical examination scenario may be generated by selecting an additionally-necessary medical examination item by accepting feedback based on the analysis result from the medical examination analysis unit when the patient inputs the answer to the generated medical examination scenario.

In the step c), the expression in each medical examination item may be converted into a familiar expression that is easy for the patient to understand by reflecting one or more of data such as age, usage language, education level, and residential area among the patient data through natural language processing of an artificial intelligence.

The step d) may include: a structuring step of classifying the patient answer to the medical examination into an item or attribute necessary for a diagnosis based on its meaning: a standardizing step of replacing the classified item and attribute with a standard word or sentence having the same meaning in standard terminology using the Korean standard terminology of medicine (KOSTOM) or systematized nomenclature of medicine clinical terms (SNOMED-CT); and an encoding step of assigning a code corresponding to the standard terminology to the standard word or sentence.

In the step e), a medical record may be generated by reconfiguring medical examination result data acquired by analyzing the answer to the medical examination scenario that is input by the patient based on a medical term.

As set forth above, the system and the method according to the present disclosure may improve the efficiency of the communication between the medical personnel and the patient by providing the medical examination for the essential items before the treatment.

The system and the method according to the present disclosure may help the medical personnel establish the accurate diagnosis and treatment plan without overlooking the important disease by selecting the medically important questions and collecting the patient answers to these questions in advance.

The system and the method according to the present disclosure may help the medical personnel quickly identify the patient details by reconfiguring and providing the patient answer to the medical examination in the form of the medical record familiar to the medical personnel.

The system and the method according to the present disclosure may minimize the human resources necessary for organizing and inputting the medical examination result by transmitting the reconfigured medical examination result in the form of the medical record to the medical personnel terminal or the electronic medical record (EMR) server.

The system and the method according to the present disclosure may help the patient to have the better understanding of the question and provide the more specific answer by selecting the medically important question based on the data such as the patient age, usage language, education level, residential area, or the like, and reconfiguring the same into the expressions that the patient may understand.

The system and the method according to the present disclosure may allow the medical consumer to have the higher satisfaction with the medical service by collecting the medically important data in advance and helping the medical personnel spend more time on the in-depth survey or consultation in the medical situation where the treatment time is limited.

The system and the method according to the present disclosure may provide the maximized interoperability and efficiency of the medical examination data for the development of the artificial intelligence, the statistical research, or the like by loading the collected medical examination results into the structuralized data based on the standard terms such as the SNOMED-CT.

### BRIEF DESCRIPTION OF THE DRAWINGS

FIG. 1 is a view for explaining an artificial intelligence medical examination and medical record generation system according to an embodiment of the present disclosure.

FIG. 2 is a view showing a configuration of the artificial intelligence medical examination and medical record generation system according to an embodiment of the present disclosure.

FIG. 3 is a view for explaining structuralizing, standardizing, and encoding of an answer to medical examination according to an embodiment of the present disclosure.

FIG. 4 is a view for explaining a medical examination scenario according to an embodiment of the present disclosure.

FIG. 5 is a view for explaining a structure of a medical record according to an embodiment of the present disclosure.

FIG. 6 is a view showing an artificial intelligence medical examination and medical record generation method according to another embodiment of the present disclosure.

### DETAILED DESCRIPTION OF THE EMBODIMENTS

Various advantages and features of the present disclosure and methods accomplishing the same are apparent from embodiments described below in detail with reference to the accompanying drawings.

However, the present disclosure is not limited to the embodiments described below, and may be implemented in various different forms.

These embodiments in the specification are provided only to make the present disclosure complete and allow those skilled in the art to which the present disclosure pertains to completely appreciate the scope of the present disclosure.

In addition, the present disclosure is defined by the scope of the claims.

Therefore, in some embodiments, well-known components, well-known operations, and well-known techniques are not described in detail in order to avoid ambiguous interpretation of the present disclosure.

In addition, like reference numerals throughout the specification denote like elements, and terms used (or referred to) in the specification are provided for describing the embodiments and are not intended to limit the present disclosure.

In the specification, a term of a singular number may include its plural number unless specifically indicated otherwise in the context, and components and operations referred to as being “included (or provided)” do not exclude the presence or addition of one or more other components and operations.

Unless defined otherwise, all terms (including technical and scientific terms) used in the specification have the same meaning as meanings commonly understood by those skilled in the art to which the present disclosure pertains.

In addition, terms generally used as defined in a dictionary are not to be interpreted as having ideal or excessively formal meanings unless clearly indicated otherwise.

Hereinafter, the embodiments of the present disclosure are described with reference to the accompanying drawings.

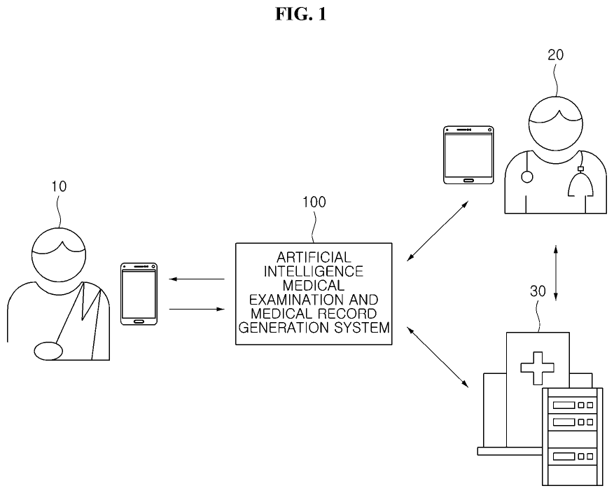

FIG. 1 is a view for explaining an artificial intelligence medical examination and medical record generation system according to an embodiment of the present disclosure.

An artificial intelligence medical examination and medical record generation system 100 according to the present disclosure may select or generate an appropriate medical examination item among medical examination items pre-stored in a medical examination database (DB) 140 based on patient data and provides a patient 10 with the selected or generated appropriate medical examination item.

The patient 10 may register as a member of the artificial intelligence medical examination and medical record generation system 100 prior to using the system according to the present disclosure. The patient 10 may fill in the patient data when registering as the member, where the patient data may include data on patient age, gender, usage language, education level, residential area, past treatment history, allergy, or the like. The treatment history may exist in a medical institution using an electronic medical record (EMR) system linked to the patient 10. In this case, the system 100 may receive data stored in an EMR server 30 of an electronic medical record (EMR) based on consents of the medical institution and the patient 10 and utilize the received data as the patient data.

The artificial intelligence medical examination and medical record generation system 100 according to the present disclosure may analyze the patient data through a medical examination-generation artificial intelligence, generate a medical examination scenario by selecting the appropriate medical examination item among the medical examination items pre-stored in the medical examination DB 140, provide the patient 10 with the generated medical examination scenario, and then receive and analyze an answer to the medical examination scenarios to generate a medical record.

In the present disclosure, the medical examination scenario may include a question that leads to a specific answer to a patient's symptom, rather than a simple yes/no answer to the question. In addition, the system 100 may use a familiar expression used in everyday life for the patient 10 to feel just like when the patient consults with a real doctor, thereby helping the patient 10 better understand the question and comfortably perform the medical examination.

To this end, the artificial intelligence medical examination and medical record generation system 100 according to the present disclosure may convert each medical examination scenario item into the familiar expression that is easy for the patient 10 to understand through natural language processing of the medical examination artificial intelligence based on the data such as the age, usage language, education level, and residential area of the patient 10.

For example, the system 100 may determine whether to directly provide the medical term, to provide additional commentary on a specialized term in particular, or to provide all terms in an easy-to-understand form, depending on the age or education level of the patient 10. In addition, for example, an expression method of the same symptom may vary depending on the residential area or usage language of the patient 10. Accordingly, the system 100 may distinguish an option expressed in standard language, dialect, or foreign language, and then provide the same.

In addition, the artificial intelligence medical examination and medical record generation system 100 according to the present disclosure may load medical examination data for the data to be easily interoperated with another database such as the electronic medical record (EMR) by configuring each medical examination scenario based on standard terminology such as the ‘Korean standard terminology of medicine (KOSTOM)’ and ‘systematized nomenclature of medicine clinical terms (SNOMED-CT)’.

The artificial intelligence medical examination and medical record generation system 100 according to the present disclosure may systematically analyze the answer to the medical examination scenario that is received from the patient 10 through structuralizing, standardizing, and encoding processes, and then a reconfiguring process of the same into the form of a treatment chart familiar to medical personnel 20 to thus generate the medical record.

In addition, the artificial intelligence medical examination and medical record generation system 100 according to the present disclosure may transmit the generated medical record to the medical personnel 20 or the EMR server 30 of the electronic medical record (EMR) system accessed by the medical personnel.

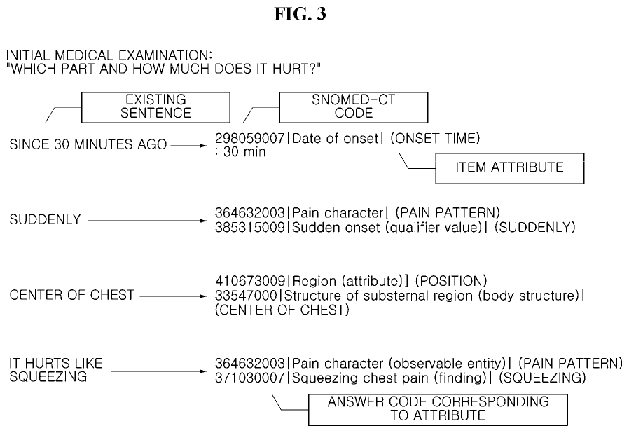

Methods for structuralizing, standardizing, and encoding the answer by analyzing the answer to the medical examination scenarios are described in detail with reference to FIGS. 3 to 5.

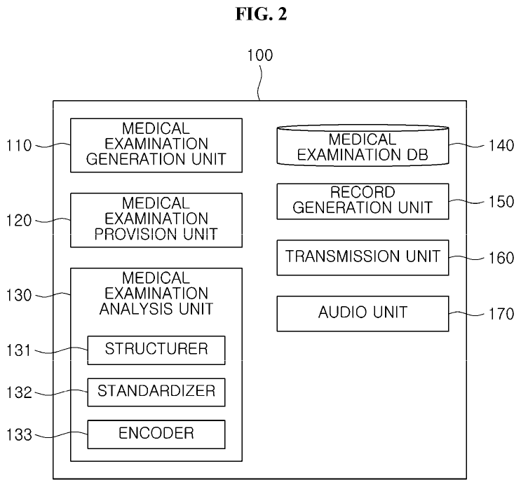

FIG. 2 is a view showing a configuration of the artificial intelligence medical examination and medical record generation system according to an embodiment of the present disclosure.

The artificial intelligence medical examination and medical record generation system 100 according to the present disclosure may include a medical examination generation unit 110, a medical examination provision unit 120, a medical examination analysis unit 130, the medical examination DB 140, a record generation unit 150, a transmission unit 160, and an audio unit 170.

The medical examination generation unit 110 may generate or update the medical examination scenario by selecting the item appropriate for the patient.

That is, the medical examination generation unit 110 may generate the medical examination scenario by selecting the appropriate item among the medical examination items stored in the medical examination DB 140 through the medical examination-generation artificial intelligence based on the previously-input patient data and symptom. In addition, in case that the patient data exists in the EMR server 30 of the linked electronic medical record (EMR), the medical examination generation unit 110 may receive and reflect the same to thus generate the medical examination scenario.

Here, the patient may complete the answer to the generated medical examination scenario and the medical examination analysis unit 130 may thus generate an analysis result. In this case, the medical examination generation unit 110 may receive this result in the form of feedback, select an additionally-necessary medical examination item again, and update the medical examination scenario. In addition, the medical examination generation unit 110 may update the medical examination item in consideration of a corrected content when the patient data or data acquired by structuralizing an existing medical examination item and an answer thereto is corrected.

The medical examination providing unit 120 may convert the medical examination scenario generated by the medical examination generation unit 110 into the expression familiar to the patient, and transmit and provide the same to a patient terminal.

In more detail, the medical examination provision unit 120 may convert an expression in each medical examination item into an expression familiar and natural to the patient by reflecting the data such as the patient age, usage language, education level, and residential area through the natural language processing of a medical examination-provision artificial intelligence, and transmit the reconfigured medical examination scenario to the patient terminal.

The medical examination analysis unit 130 may analyze the answer to the medical examination scenario that is received from the patient.

Here, the medical examination analysis unit 130 may be divided into a structurer 131, a standardizer 132, and an encoder 133 based on their functions.

The structurer 131 may classify each answer to the medical examination item into an item or attribute necessary for a diagnosis based on its meaning.

The standardizer 132 may replace the item or attribute classified by the structurer 131 with a word or a sentence having the same meaning in the standard terminology using the KOSTOM or the SNOMED-CT.

The encoder 133 may assign a code corresponding to the standard terminology to the data standardized by the standardizer 132.

Detailed descriptions of the structurer 131, standardizer 132, and encoder 133 of the medical examination analysis unit 130 are provided with reference to FIGS. 3 to 5.

The medical examination DB 140 may store data on the medical examination item and a patient's past answer thereto, and the medical examination item may be newly generated or updated by the function of the medical examination generation unit 110.

The record generation unit 150 may generate the medical record based on a medical examination result analyzed by the medical examination analysis unit 130.

Here, the medical record may be generated by reconfiguring the structuralized, standardized, and encoded medical examination results into the treatment chart based on the medical term familiar to the medical personnel 20.

The transmission unit 160 may transmit the medical record generated by the record generation unit 150 to a terminal carried by the medical personnel 20 or the EMR server 30 of the electronic medical record (EMR) system to which the medical personnel have access rights.

The artificial intelligence medical examination and medical record generation system 100 according to the present disclosure may convert each medical examination item into voice and provide the same for a patient who cannot understand the medical examination scenario, such as an illiterate or a blind person. To this end, the system 100 may further include the audio unit 170.

Here, the audio unit 170 may convert the medical examination scenario in which expression reconfiguration is completed based on a patient background by the medical examination provision unit 120 into audio and provides the same to the patient. In addition, the audio unit 170 may adopt a conversion method based on the accent or tone familiar to the patient in the voice conversion, if possible.

FIG. 3 is a view for explaining structuralizing, standardizing, and encoding of the answer to the medical examination according to an embodiment of the present disclosure.

The patient may answer, “I suddenly feel a squeezing pain in the center of the chest since 30 minutes ago” to a question “Which part and how much does it hurt?” generated by the artificial intelligence medical examination and medical record generation system 100 according to the present disclosure. In this case, contents of these question and answer may be analyzed using the structuralizing, the standardizing, and the encoding as follows.

First of all, the system 100 may structuralize the content of the answer into a pain-onset time, a pain pattern, and a pain position through the structuralizing process of analyzing the answer by meaning and classifying the same based on the item or attribute necessary for the diagnosis.

The patient answer to the structuralized attribute may be replaced with the word or sentence having the same meaning in the standard terminology. In this case, the system 100 may standardize “suddenly” and “since 30 minutes ago” as the pain-onset time and “squeezing pain” as the pain pattern, and “the center of the chest” as the pain position.

In addition, the system 100 may assign a code corresponding to the SNOMED-CT to the standardized word or sentence. In this case, the system 100 may assign 298059007 to the pain-onset time, 364632003 to the pain pattern, 410673009 to the pain position, 385315009 to “suddenly”, 371030007 to “squeezing pain”, and 33547000 to “the center of the chest”.

The artificial intelligence medical examination and medical record generation system 100 according to the present disclosure may structuralize, standardize, and encode the patient answer in this way and store the same in the medical examination DB 140, thereby greatly improving efficiency of the medical examination data difficult for secondary use such as a statistical analysis or an artificial intelligence development due to limitation of the unstructured data despite its great importance in a clinical decision-making process.

When structuralizing and standardizing the medical examination item and the medical examination result data loaded in the medical examination DB 140, the artificial intelligence medical examination and medical record generation system 100 according to the present disclosure may utilize Korean standard classification of diseases (KCD) commonly used for diagnosis names or the like in the electronic medical record (EMR), international classification of diseases (ICD), or the like along with the SNOMED-CT which are international standard medical terms and the KOSTOM which is a Korean standard medical terminology developed by the Ministry of Health and Welfare in Korea.

The medical examination DB 140 of the artificial intelligence medical examination and medical record generation system 100 according to the present disclosure may utilize a database structure enabling the database to accommodate the unstructured data such as elastic search/mongo DB. The medical examination item loaded into the DB 140 may be generated through inspecting and structuralizing processes of a necessary medical examination content that is performed by a medical expert.

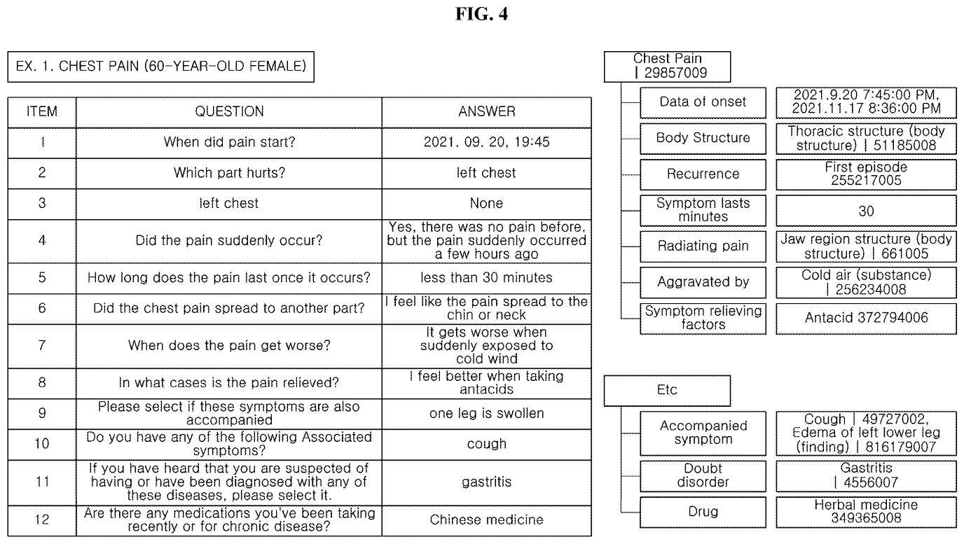

FIG. 4 is a view for explaining the medical examination scenario according to an embodiment of the present disclosure.

For example, a 60-year-old female may complain of her chest pain and use the artificial intelligence medical examination and medical record generation system 100 of the present disclosure. In this case, as shown in FIG. 4, the system 100 may perform the medical examination by configuring a scenario with 12 categories (or items).

The system 100 may then analyze answers to 12 items and structuralize, standardize, and encode the answers as described above.

The number of items in clinical performance examination (CPX) of the national examination for doctors is 54 (as of year of 2018), which is selected to cover treatment scenarios for patient symptoms that the doctors often encounter in primary care.

In addition, when the main symptoms complained of by the patient while visiting the clinic are classified based on ICD-10 which is the international standard disease classification system, the symptoms may be broadly categorized into a total of 93 items according to ‘Main symptom, diagnosis, and death analysis of 230,000 patients admitted to Seoul National University hospital’ (Jin Ho-Jun & Kim Seong-Kwon, Journal of the Korean Society of Internal Medicine, Vol. 65: No. 5, 2003). In addition, analysis results of the chief complaints of patients who visit an emergency medical center based on the ICD code may be categorized into a total of 82 items according to ‘A study on the distribution of main symptoms and main diagnoses of patients visiting emergency medical centers’ (Lee Kyung-Sook, Digital Policy Research, Vol. 10: No. 10, 2012).

Considering the above facts and studies, it is required for a doctor in charge of the primary care in a clinic or an emergency room to be able to respond to at least around 90 symptoms. Therefore, in order to satisfy these medical needs and provide more subdivided medical examinations for each patient group, the artificial intelligence medical examination and medical record generation system 100 according to the present disclosure may provide 100 or more medical examination scenarios based on the above studies. For example, abdominal pain in an adult and abdominal pain in a child are different from each other in their disease groups requiring differential diagnoses, and accordingly, their medical examination items and processes also need to be different from each other.

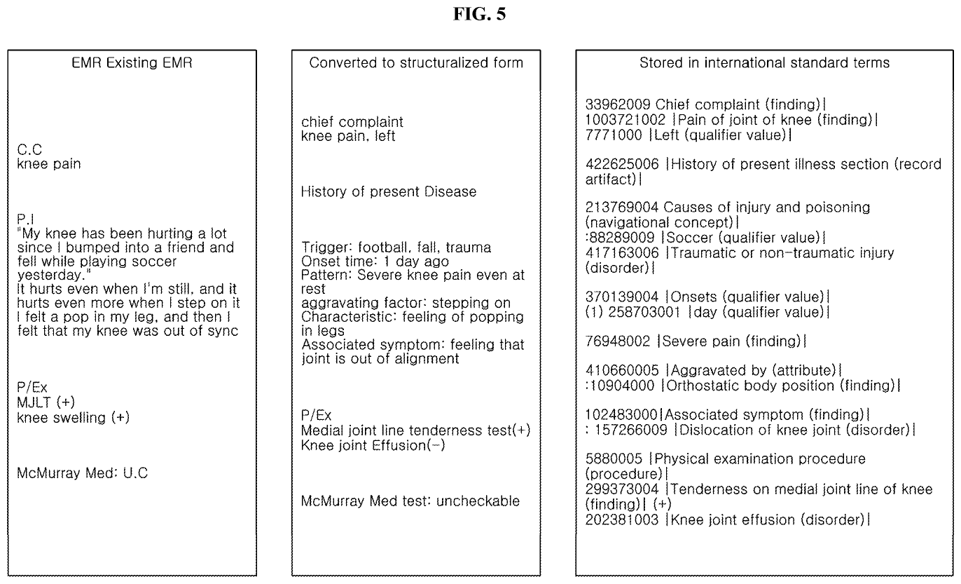

FIG. 5 is a view for explaining the treatment chart according to an embodiment of the present disclosure.

The medical record generated from the artificial intelligence medical examination and medical record generation system 100 according to the present disclosure may be generated by analyzing the patient answer to the medical examination scenario through the structuralizing, standardizing, and encoding processes, and then the reconfiguring process of the same into the medical record based on the medical term that is easy for the medical personnel to understand.

The artificial intelligence medical examination and medical record generation system 100 according to the present disclosure may structuralize, standardize, encode and load all components that may be used in the medical record loaded into the existing EMR, such as symptoms and diseases, drugs, and body parts, based on 923,182 cases in a SNOMED-CT database and 320,993 cases in a KOSTOM database. The primary structuring, standardizing, and encoding of the data may be performed using the analysis artificial intelligence of the present disclosure. However, a high degree of accuracy is required due to nature of the medical care, and the data on which the above work is completed may go through reviewing and correcting processes performed by a group of experts including doctors, nurses, or the like.

Based on these standardized components, the system 100 may reconfigure the patient answer to the medical examination scenarios that is structuralized through the analysis artificial intelligence into a form of the medical record. Here, the system 100 may help the medical personnel conveniently refer to the answer by further performing a converting process of a standardized term into a general medical term that is easy for the medical personnel to understand, rather than directly providing the medical personnel with the standardized term.

Meanwhile, even though the medical record displayed to the medical personnel uses the general medical term, the system 100 may provide the standardized and encoded data when storing the data in the EMR server 30 or transmitting the data for the secondary use, or the like to thus maximize the interoperability and efficiency of the data.

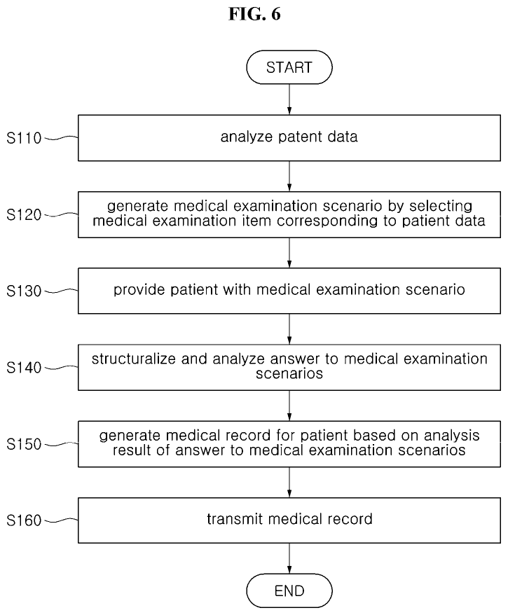

FIG. 6 is a view showing an artificial intelligence medical examination and medical record generation method according to another embodiment of the present disclosure.

The method may include analyzing data of a patient who wants to use an artificial intelligence medical examination and medical record generation system (S110).

The patient who wants to use the artificial intelligence medical examination and medical record generation system 100 according to the present disclosure may provide patient data to the system by registering as its member.

The artificial intelligence medical examination and medical record generation system 100 may analyze data on the age, gender, usage language, education level, residential area, past treatment history, allergy, or the like of the patient based on his/her member data.

A record matching the member data may exist in an electronic medical record (EMR) server 30 of a linked medical institution. In this case, the artificial intelligence medical examination and medical record generation system 100 may receive and analyze additional patient data from the EMR server 30 based on consents of the patient and the medical institution.

The method may include generating a medical examination scenario by selecting an appropriate medical examination item based on the patient data (S120).

The artificial intelligence medical examination and medical record generation system 100 according to the present disclosure may select the medical examination item appropriate for the patient based on the analyzed patient data and symptom, and generate the medical examination scenario by integrating the items.

The method may include providing the patient with the medical examination scenario (S130), that is, reconfiguring the medical examination scenario generated in Step S120 and providing the patient with the reconfigured scenario.

The artificial intelligence medical examination and medical record generation system 100 according to the present disclosure may use the analyzed patient data to reconfigure each medical examination scenario item into a familiar expression that is easy for the patient to understand and provide the same to the patient.

The method may include analyzing an answer to the medical examination scenario that is received from the patient (S140). As described above, the system 100 may analyze the answer to the medical examination scenarios through structuralizing, standardizing, and encoding processes.

The method may include generating a medical record in an expression familiar to medical personnel based on the above analysis result (S150).

The generated medical record may include data on a main symptom such as the onset time, duration, and pattern of the main symptom, data on an associated symptom, data on a past history such as past treatment and surgery history, data on a family history such as family medical history, and data on vitality such as drinking status or lifestyle along with the main symptom, age and gender of the patient.

The method may include transmitting the generated medical record (S160), that is, directly providing the medical record to a terminal used by medical personnel 20 or transmitting the medical record to the EMR server 30 of a linked EMR system to which the medical personnel have access rights.

The present disclosure is not limited to the above-mentioned specific embodiments, and may be variously modified by those skilled in the art to which the present disclosure pertains without departing from the scope and spirit of the present disclosure as claimed in the accompanying claims. These modifications should also be understood to fall within the scope and spirit of the present disclosure as claimed in the following claims.

What is claimed is:

1. An artificial intelligence medical examination and medical record generation system, the system comprising:
a medical examination database (DB) storing a medical examination item and a past medical examination result;
a medical examination generation unit configured for generating or updating a medical examination scenario by selecting an appropriate medical examination item through a medical examination-generation artificial intelligence based on patient data;
a medical examination provision unit configured for processing and providing the medical examination scenario with an expression familiar to a patient through natural language processing of a medical examination-provision artificial intelligence based on the patient data;
a medical examination analysis unit configured for analyzing a patient answer to the medical examination scenario through structuralizing, standardizing, and encoding processes;
a record generation unit configured for generating a medical record familiar to medical personnel based on an analysis result generated by the medical examination analysis unit; and
a transmission unit configured for transmitting the medical record to the medical personnel and an electronic medical record (EMR) system.

2. The system of claim 1, wherein the medical examination analysis unit includes:
a structurer configured for classifying the answer to the medical examination item into an item or attribute necessary for a diagnosis based on its meaning;
a standardizer configured for replacing the classified item and attribute with a standard word or sentence having the same meaning in standard terminology using Korean standard terminology of medicine (KOSTOM) or systematized nomenclature of medicine clinical terms (SNOMED-CT); and
an encoder configured for assigning a code corresponding to the standard terminology to the standard word or sentence.

3. The system of claim 1, wherein the medical examination generation unit is configured to generate the medical examination scenario by selecting the appropriate medical examination item among the stored medical examination items based on the patient data and a patient symptom.

4. The system of claim 1, wherein the patient data includes data on a past treatment history of the patient, and one or more of age, gender, usage language, education level, past treatment history, residential area, lifestyle, or allergy data, input by the patient before medical examination is generated, and
the medical examination generation unit is configured to update the medical examination item in consideration of a corrected content when the patient data or data acquired by structuralizing an existing medical examination item and an answer to the medical examination item is corrected.

5. The system of claim 1, wherein the medical examination generation unit is configured to generate the updated medical examination scenario by selecting an additionally-necessary medical examination item based on the analysis result generated by the medical examination analysis unit when the patient inputs the answer to the medical examination scenario.

6. The system of claim 1, wherein the medical examination provision unit is configured to convert an expression in the medical examination scenario into a familiar expression that is easy for the patient to understand through the natural language processing based on the patient data.

7. The system of claim 1, wherein the record generation unit is configured to generate the medical record by converting the patient answer to the medical examination scenario into an expression that is easy for the medical personnel to understand based on the analysis performed by the medical examination analysis unit.

8. The system of claim 1, wherein the transmission unit is configured to directly transmits the medical record to a medical personnel terminal or to an EMR server of the linked EMR system to which the medical personnel have access rights.

9. An artificial intelligence medical examination and medical record generation method, the method comprising:
analyzing data of a patient who wants to use an artificial intelligence medical examination and medical record generation system;
generating a medical examination scenario by selecting a medical examination item corresponding to the patient data;
reconfiguring the medical examination scenario into an expression corresponding to the patient data and providing the expression to the patient;
systematizing and analyzing an answer to the medical examination scenario that is received from the patient;
generating a medical record for the patient based on an analysis result of the answer to the medical examination scenario; and
transmitting the medical record to a medical personnel terminal or a linked electronic medical record (EMR) server.

10. The method of claim 9, wherein the patient data includes data on a past treatment history of the patient, and one or more of age, gender, usage language, education level, past treatment history, residential area, lifestyle, or allergy data, and
in the generating of the medical examination scenario, the medical examination item is corrected or updated in consideration of a corrected content when the patient data or data acquired by structuralizing an existing medical examination item and an answer to the existing medical examination item is corrected.

11. The method of claim 9, wherein, in the generating of the medical examination scenario, the updated medical examination scenario is generated by selecting an additionally-necessary medical examination item by accepting feedback based on the analysis result from the medical examination analysis unit when the patient inputs the answer to the generated medical examination scenario.

12. The method of claim 9, wherein, in the reconfiguring of the medical examination scenario, the expression in each medical examination item is converted into a more familiar expression that is easier for the patient to understand by reflecting one or more of data such as age, usage language, education level, and residential area among the patient data through natural language processing of an artificial intelligence.

13. The method of claim 9, wherein the systematizing and analyzing of the answer comprises:
classifying the patient answer to the medical examination into an item or attribute necessary for a diagnosis based on its meaning;
replacing the classified item and attribute with a standard word or sentence having the same meaning in standard terminology using Korean standard terminology of medicine (KOSTOM) or systematized nomenclature of medicine clinical terms (SNOMED-CT); and
assigning a code corresponding to the standard terminology to the standard word or sentence.

14. The method of claim 9, wherein, in the generating of the medical record, the medical record is generated by reconfiguring medical examination result data acquired by analyzing the answer to the medical examination scenario that is input by the patient based on a medical term.
